(19) United States
(12) Patent Application Publication
(10) Pub. No.: US 2026/0102410 A1
(43) Pub. Date: Apr. 16, 2026

(54) Treatment of Alcoholic Hepatitis

(71) Applicant: Durect Corporation, Cupertino, CA (US)

(72) Inventors: Weiqi Lin, Emerald Hills, CA (US); James E. Brown, Los Gatos, CA (US); Terrence Blaschke, Stanford, CA (US)

(21) Appl. No.: 19/364,999

(22) Filed: Oct. 21, 2025

Related U.S. Application Data
(63) Continuation of application No. 17/765,202, now abandoned.
(60) Provisional application No. 62/908,465, filed on Sep. 30, 2019.
(60) Provisional application No. 62/933,206, filed on Nov. 8, 2019.
(60) Provisional application No. 63/060,564, filed on Aug. 3, 2020.
(60) Provisional application No. 63/081,208, filed on Sep. 21, 2020.

(51) Int. Cl. A61K 31/575; A61K 9/00; A61K 31/047; A61P 1/16

(52) U.S. Cl. CPC A61K 31/575; A61K 9/0019; A61K 31/047; A61P 1/16

(57) ABSTRACT

Methods of treating alcoholic hepatitis (AH) are provided. For instance, the methods may comprise administering 5-cholesten-3,25-diol, 3-sulfate (25HC3S) or a salt thereof.

### BACKGROUND

Alcoholic hepatitis (AH) is an alcoholic liver disease caused by long-term intake of alcohol (ethanol). Signs and symptoms of AH include acute jaundice, fever and weight loss, as well as several complications such as ascites and hepatic encephalopathy, leading to hepatic cirrhosis. AH is associated with a high mortality burden, up to 15% at 30 days, which is dependent on disease severity at presentation. The 1-month mortality rate of severe AH is as high as 30%-50%.

Corticosteroids are generally used to treat severe cases of AH. However, treatment with corticosteroids shows conflicting results, is associated with an increased risk of infection, and is not suitable for patients with gastrointestinal bleeding or for patients who have allergy to steroids. Corticoid steroids are typically taken daily in an amount such as 30 mg to 40 mg, depending on the specific corticosteroid used. Patient compliance tends to be low as patients often do not take their medication when they should.

Liver transplantation is a curative treatment option for AH, but patients must abstain from alcohol use for 6 months to qualify. But the 6-month-mortality of severe AH is high (approximately 40%). While liver transplantation is a treatment option, severe AH patients often die before meeting the transplantation criteria.

U.S. Pat. No. 8,399,441, which is incorporated by reference herein, discloses the use of 5-cholesten-3,25-diol, 3-sulfate (25HC3S) and salts thereof for the treatment of conditions associated with high cholesterol and/or high triglycerides and/or inflammation (e.g., hypercholesterolemia, hypertriglyceridemia, non-alcoholic fatty liver diseases, atherosclerosis, etc.).

U.S. Pat. No. 9,034,859, which is incorporated by reference herein, discloses the use of 25HC3S and salts thereof for prevention and treatment of liver damage or disease.

U.S. Pat. No. 10,272,097, which is incorporated by reference herein, discloses oxygenated cholesterol sulfates, e.g., 25HC3S and salts thereof, for preventing and/or treating ischemia, organ dysfunction and/or organ failure, including multiple organ dysfunction syndrome (MODS), and necrosis and apoptosis associated with organ dysfunction/failure.

ClinicalTrials.gov includes disclosure of a research study to assess the safety, pharmacokinetics and pharmacodynamics of 25HC3S in patients with AH, with dose escalation including three doses: 30 mg, 90 mg, and 150 mg. See Clinical Trials.gov Identifier: NCT03432260.

There is an urgent need for improved methods to treat AH.

### SUMMARY

The present disclosure provides a variety of methods of treating alcoholic hepatitis (AH). The methods involve administering an effective amount of 5-cholesten-3,25-diol, 3-sulfate (25HC3S) or salt thereof. In certain instances, the methods involve administering an effective amount of 5-cholesten-3,25-diol, 3β-sulfate or salt thereof.

The results of the present disclosure are surprising for several reasons. For instance, in some embodiments, the low dose of the present disclosure is surprising. Further, in some embodiments, the low dose frequency is surprising. Still further, in some embodiments, the low number of doses to attain efficacy in the treatment of AH is surprising. The results of the present disclosure are also surprising to the extent that the low dose amount, low dose frequency, and low total number of doses can provide results that are similar to or better than corticosteroids which require larger dose amounts, more frequent dosing, and/or a higher total number of doses. Still further, the present disclosure is also capable of achieving efficacious results in AH patients characterized by particular diagnostic criteria, such as Model for End-stage Liver Disease (MELD) score and/or other criteria discussed elsewhere herein.

Further aspects of the disclosure:
1. A method of treating alcoholic hepatitis in a human subject in need thereof, the method comprising administering to the subject 5-cholesten-3,25-diol, 3-sulfate (25HC3S) or salt thereof in an amount that is sufficient to treat the alcoholic hepatitis.
2. The method according to aspect 1, wherein the administering comprises administering a total amount of the 25HC3S or salt thereof ranging from about 1 mg to about 1000 mg.
3. The method according to aspect 1 or 2, wherein the administering comprises administering a total amount of the 25HC3S or salt thereof ranging either: (a) from about 10 mg/month to about 500 mg/month; or (b) from about 10 mg/month to about 400 mg/month.
4. The method according to any one of aspects 1 to 3, the method comprising administering to the human subject a total amount of either: (a) about 10 mg/month to about 400 mg/month; or (b) about 10 mg to about 400 mg of 25HC3S or salt thereof in a period of one month; wherein:
   the total amount is sufficient to treat the alcoholic hepatitis;
   the total amount is administered in one or more separate doses comprising at least a first dose; and
   the period of one month is measured from the beginning of administration of the first dose.
5. The method of any one of aspects 2 to 4, wherein the total amount is administered in from one to fifteen separate doses.
6. The method of any one of aspects 2 to 4, wherein the total amount is administered in from one to five separate doses.
7. The method of any one of aspects 2 to 4, wherein the total amount is administered in one dose or in two separate doses, preferably as a single dose.
8. The method of any one of aspects 2 to 7, wherein the total amount is at least about 20 mg.
9. The method of any one of aspects 2 to 8, wherein the total amount is not more than about 300 mg.
10. The method of any one of aspects 2 to 8, wherein the total amount is not more than about 200 mg.
11. The method of any one of aspects 2 to 8, wherein the total amount is not more than about 140 mg.
12. The method of any one of aspects 4 to 11, wherein at least one, and preferably each, of the one or more separate doses comprises from about 10 mg to about 200 mg of 25HC3S or salt thereof.
13. The method of any one of aspects 4 to 12, wherein at least one, and preferably each, of the one or more separate doses comprises from about 10 mg to about 120 mg of 25HC3S or salt thereof.
14. The method of any one of aspects 4 to 13, wherein at least one, and preferably each, of the one or more separate doses comprises from about 20 mg to about 100 mg of 25HC3S or salt thereof.
15. The method of any one of aspects 2 to 14, wherein the total amount is administered in two or more separate doses that are administered at a dose frequency ranging from daily to once every week.
16. The method of aspect 15, wherein the dose frequency ranges from once every two days to once every week.
17. The method of aspect 15, wherein the dose frequency ranges from once every two days to once every five days.
18. The method of aspect 15, wherein the dose frequency ranges from once every two days to once every four days.
19. The method of aspect 4, wherein:
   the total amount is administered in one dose or in two separate doses;
   the one dose comprises from about 10 mg to about 200 mg of 25HC3S or salt thereof;
   each of the two separate doses comprises from about 10 mg to about 200 mg of 25HC3S or salt thereof; and
   the two separate doses are administered at a dose frequency ranging from once every two days to once every four days.
20. The method of aspect 19, wherein the total amount is administered in one dose and wherein the one dose comprises: (a) from about 20 mg to about 40 mg of 25HC3S or salt thereof; or (b) from about 80 mg to about 100 mg of 25HC3S or salt thereof: or (c) from about 140 mg to about 160 mg of 25HC3S or salt thereof.
21. The method of aspect 19, wherein the total amount is administered in two separate doses and wherein each of the two separate doses comprises: (a) from about 20 mg to about 40 mg of 25HC3S or salt thereof; or (b) from about 80 mg to about 100 mg of 25HC3S or salt thereof; or (c) from about 140 mg to about 160 mg of 25HC3S or salt thereof.
22. The method of any one of aspects 4 to 21, wherein a total amount per kg of 25HC3S or salt thereof that is administered in the period ranges from about 0.05 mg/kg to about 20 mg/kg.
23. The method of aspect 22, wherein the total amount per kg ranges from about 0.1 mg/kg to about 10 mg/kg.
24. The method of aspect 22, wherein the total amount per kg ranges from about 0.2 mg/kg to about 6 mg/kg.
25. The method of aspect 22, wherein the total amount per kg ranges from about 0.3 mg/kg to about 1.5 mg/kg.
26. The method of any one of aspects 1 to 25, wherein the administering is performed parenterally, intravenously, intramuscularly, or subcutaneously.
27. The method of any one of aspects 1 to 26, wherein the administering is performed by injection.
28. The method of any one of aspects 1 to 27, wherein the 25HC3S or salt thereof is administered in a formulation comprising the 25HC3S or salt thereof and a pharmaceutically acceptable carrier.
29. The method of any one of aspects 1 to 28, wherein the 25HC3S or salt thereof comprises a salt of 25HC3S, wherein the salt of 25HC3S is preferably the sodium salt.
30. The method of any one of aspects 4 to 29, wherein the one or more separate doses are administered over an administration period of not more than 14 days.
31. The method of any one of aspects 1 to 30, wherein the human subject has a Model for End-stage Liver Disease (MELD) score of at least 11.
32. The method of any one of aspects 1 to 31, wherein the human subject has a MELD score ranging from 11 to 45.
33. The method of any one of aspects 1 to 32, wherein the human subject has a MELD score ranging from 15 to 40.
34. The method of any one of aspects 1 to 33, wherein the human subject has a MELD score of at least 20.
35. The method of any one of aspects 1 to 34, wherein the human subject has a MELD score of at least 30.
36. The method of any one of aspects 1 to 35, wherein the human subject has a Maddrey's discriminant function (MDF) of at least 32.
37. The method of any one of aspects 1 to 36, wherein the alcoholic hepatitis is characterized by reduced liver function that occurs within 13 weeks of onset.
38. The method of any one of aspects 1 to 36, wherein the alcoholic hepatitis is characterized by reduced liver function that occurs within 10 weeks of onset.
39. The method of any one of aspects 1 to 38, wherein the alcoholic hepatitis is characterized by reduced liver function such that the subject exhibits a reduced rate of clearance of 25HC3S as compared to a subject that does not have alcoholic hepatitis.
40. The method of aspect 39, wherein the alcoholic hepatitis is characterized by reduced liver function such that the subject exhibits a rate of clearance of 25HC3S that is 50% or less, 40% or less, 30% or less, or 25% or less of the rate of clearance of 25HC3S from the plasma by a subject that does not have alcoholic hepatitis.
41. The method of any one of aspects 1 to 40, wherein the alcoholic hepatitis is characterized by reduced liver function such that the subject exhibits a half-life time of 25HC3S in the plasma after administration (T_{1/2}) that is 1.5-fold greater or more, or 2-fold greater or more, as compared to a subject that does not have alcoholic hepatitis.
42. The method of any one of aspects 1 to 41, wherein the subject exhibits a half-life time of 25HC3S in the plasma after administration (T_{1/2}) ranging from about 1.5 hours to about 6 hours or from about 2 hours to about 5 hours.
43. The method of any one of aspects 1 to 42, wherein the subject exhibits a Cmax of 25HC3S ranging from about 500 ng/mL to about 10,000 ng/mL, from about 600 ng/ml to about 7000 ng/mL, or from about 700 ng/mL to about 5000 ng/ml.
44. The method of any one of aspects 1 to 43, wherein the subject exhibits a Cmax of 25HC3S ranging from about 500 ng/mL to about 10,000 ng/mL, from about 600 ng/mL to about 7000 ng/mL, or from about 700 ng/mL to about 5000 ng/ml, per 100 mg of intravenously administered 25HC3S or salt thereof.
45. The method of any one of aspects 1 to 44, wherein the subject exhibits an AUCinf of 25HC3S ranging from about 3000 ng*h/mL to about 50,000 ng*h/mL, about 4000 ng*h/mL to about 40,000 ng*h/mL, or from about 5000 ng*h/mL to about 30,000 ng*h/mL.
46. The method of any one of aspects 1 to 45, wherein the subject exhibits an AUCinf of 25HC3S ranging from about 3000 ng*h/mL to about 50,000 ng*h/mL, about 4000 ng*h/mL to about 40,000 ng*h/mL, or from about 5000 ng*h/mL to about 30,000 ng*h/mL, per 100 mg of intravenously administered 25HC3S or salt thereof.
47. The method of any one of aspects 1 to 46, wherein the subject exhibits a volume of distribution of 25HC3S ranging from about 10 L to about 50 L, about 15 L to about 45 L, or from about 20 L to about 40 L.
48. The method of any one of aspects 1 to 47, wherein the subject exhibits a clearance of 25HC3S ranging from about 2 L to about 8 L/h, about 2.5 L/h to about 7.5 L/h, or from about 3 L/h to about 7 L/h.
49. The method of any one of aspects 1 to 48, wherein the alcoholic hepatitis is characterized by loss of liver function that occurs within 13 weeks of onset.
50. The method of any one of aspects 1 to 48, wherein the alcoholic hepatitis is characterized by loss of liver function that occurs within 10 weeks of onset.
51. 5-cholesten-3,25-diol, 3-sulfate (25HC3S) or salt thereof for use in a method of treating alcoholic hepatitis in a human subject in need thereof, wherein the method is as defined in any one of aspects 1 to 50.
52. Use of 5-cholesten-3,25-diol, 3-sulfate (25HC3S) or salt thereof in a method for the manufacture of a medicament for use in a method of treating alcoholic hepatitis in a human subject in need thereof, wherein the method is as defined in any one of aspects 1 to 50.

### BRIEF DESCRIPTION OF THE DRAWINGS

FIG. 1. compares standard of care treatment (left) and treatment with 5-cholesten-3,25-diol, 3-sulfate (25HC3S) (right). The standard of care patients received supportive treatment with or without corticosteroids. The 25HC3S patients received one or two doses of 25HC3S sodium salt, with each dose at an amount of 30 mg, 90 mg, or 150 mg.

FIG. 2 depicts a flow diagram for a treatment protocol for 25HC3S of subjects with moderate alcoholic hepatitis and severe alcoholic hepatitis.

FIG. 3A depicts mean plasma concentration of 25HC3S in subjects with moderate AH. FIG. 3B depicts mean plasma concentration of 25HC3S in subjects with severe AH.

FIG. 4A depicts the pharmacokinetic parameter of Cmax for 25HC3S administered to subjects with moderate alcoholic hepatitis and severe alcoholic hepatitis. FIG. 4B depicts the pharmacokinetic parameter of AUC for 25HC3S administered to subjects with moderate alcoholic hepatitis and severe alcoholic hepatitis.

FIG. 5 depicts a comparison of the pharmacokinetic parameters of 25HC3S administered to healthy subjects versus subjects with alcoholic hepatitis.

FIG. 6 depicts the Lille scores determined at Day 7 plotted against the pharmacokinetic parameter AUC of subjects administered 30 mg, 90 mg or 150 mg of 25HC3S.

### DETAILED DESCRIPTION OF THE INVENTION

Methods for treating alcoholic hepatitis (AH) are described herein. The methods include contacting the liver with 25HC3S or salt thereof. The contact generally involves administering to a human patient an amount of 25HC3S or salt thereof that is effective or sufficient to treat AH.

As discussed above, the results of the present disclosure are surprising for several reasons. For instance, in some embodiments, the low dose of the present disclosure is surprising. Further, in some embodiments, the low dose frequency is surprising. Still further, in some embodiments, the low number of doses to attain efficacy in the treatment of AH is surprising. The results of the present disclosure are also surprising to the extent that the low dose amount, low dose frequency, and/or low total number of doses can provide results that are similar to or better than corticosteroids which require larger dose amounts, more frequent dosing, and a higher total number of doses. Still further, the present disclosure is also capable of achieving efficacious results in AH patients characterized by particular diagnostic criteria, such as MELD score and/or other criteria discussed elsewhere herein.

### Definitions

The following definitions are used throughout:

“Treat” (treatment, treating, etc.) as used herein refers to administering 25HC3S or salt thereof to a human subject that: (1) already exhibits at least one symptom of AH; and/or (2) is diagnosed as having AH, such as by a trained clinical professional; and/or (3) is determined to have AH based on laboratory (e.g., molecular indicators) or clinical tests of one or more body fluids, such as blood. In certain embodiments, subjects are diagnosed as having AH by liver tissue biopsy. In other words, at least one parameter that is known to be associated with AH has been measured, detected or observed in the subject. “Treatment” of AH involves the lessening or attenuation, or in some instances, the complete eradication, of at least one symptom of AH that was present prior to or at the time of administration of 25HC3S or salt thereof. In some embodiments, treating AH according to the present disclosure is sufficient to improve laboratory or clinical indicators of AH in the subject as described in greater detail below. In certain instances, the improvement in the laboratory or clinical indicators of AH in the subject is such that the subject is considered to no longer have AH. In one example, the subject methods are sufficient to reduce the Maddrey discriminant function (MDF) score of a subject having a score≥32 to an MDF score that is <32.

“Liver dysfunction” denotes a condition or a state of health where the liver does not perform its expected function, such as where certain biological or molecular indicators are measured to be outside of normal physiologic ranges. Liver function represents the expected function of the liver within physiologic ranges. The person skilled in the art is aware of the respective function of the liver during medical examination. Liver dysfunction typically involves a clinical syndrome in which the development of progressive and potentially reversible physiological dysfunction in the liver, optionally in the absence of anatomic injuries.

“Liver failure” denotes liver dysfunction to such a degree that normal homeostasis cannot be maintained without external clinical intervention.

“Acute liver dysfunction” refers to reduced liver function that occurs rapidly—in days or weeks (e.g., within 26 weeks, within 13 weeks, within 10 weeks, within 5 weeks, within 4 weeks, within 3 weeks, within 2 weeks, within 1 week, within 5 days, within 4 days, within 3 days, or within 2 days)—usually in a person who has no pre-existing disease.

“Acute liver failure” refers to loss of liver function that occurs rapidly—in days or weeks (e.g., within 26 weeks, within 13 weeks, within 10 weeks, within 5 weeks, within 4 weeks, within 3 weeks, within 2 weeks, within 1 week, within 5 days, within 4 days, within 3 days, or within 2 days)—usually in a person who has no pre-existing disease. Acute liver failure is discussed in more detail below.

“Pharmaceutically acceptable” refers to a substance that does not interfere with the effectiveness of the biological activity of the active ingredient and is not toxic to the host to which it is administered.

### Patient Population

### Overview of Alcoholic Hepatitis

Alcoholic hepatitis (AH) can range from mild hepatitis, with abnormal laboratory tests being the only indication of disease, to severe liver dysfunction with complications such as jaundice (yellow skin caused by bilirubin retention), hepatic encephalopathy (neurological dysfunction caused by liver failure), ascites (fluid accumulation in the abdomen), bleeding esophageal varices (varicose veins in the esophagus), abnormal blood clotting and coma. In some cases, the patient has an onset of jaundice within prior 8 weeks, such as within prior 7 weeks, such as within prior 6 weeks, such as within prior 5 weeks, such as within prior 4 weeks, such as within prior 3 weeks, such as within prior 2 weeks and including where the patient has an onset of jaundice within the prior week. AH is typically reversible if the patient stops drinking, but hepatitis usually takes several months to resolve. AH can lead to liver scarring and cirrhosis. The typical findings on liver histology include hepatocellular necrosis and ballooning degeneration, and alcoholic Mallory's hyaline bodies (abnormal aggregations of cellular intermediate filament proteins indicative of fibrosis). Cholestasis is typically prominent. Severity of the disease can be classified according to Maddrey's discriminant function (MDF) (based on bilirubin and prothrombin time), Glasgow alcoholic hepatitis score (based on age, white blood cell count, urea, prothrombin time and bilirubin) or Model for End Stage Liver Disease (MELD) score (based on creatinine, bilirubin, and international normalized ratio for prothrombin time (INR)) (Lucey et al., N. Engl. J. Med., 360 (26), 2758-2769 (2009); Vergis et al., Gastroenterology, 152 (5): 1068-1077 (2017)). AH is classified as severe when the MDF is >32.

### Alcohol Consumption

In some cases, the patient has a history of heavy alcohol abuse of >40 g/day in females or >60 g/day in males for a minimum period of 6 months, e.g., with <60 days (e.g., <8 weeks) of abstinence before the onset of jaundice. Excess alcohol intake over many years can lead to alcoholic liver disease and AH. In some cases, the excess alcohol intake involves alcohol intake of >80 g/day for males or >60 g/day for females. Accordingly, one embodiment is the use of 25HC3S or salt thereof in treating AH in a male or female subject consuming excess alcohol, wherein the male subject is consuming >80 g of alcohol per day or the female subject is consuming >60 g of alcohol per day. In other embodiments, the present disclosure is directed to the use of 25HC3S or salt thereof in treating or alleviating AH in a male or female subject having a history of consuming excess alcohol, such as where the subject has experienced one or more times where the subject has consumed on average 40 g/day or more for 6 months or more. In certain embodiments, the subject has experienced one or more times where the subject has consumed on average 60 g/day or more for 6 months or more.

### Bilirubin

AH is characterized by elevated bilirubin, which reflects impaired metabolic function of the liver in the absence of biliary obstruction. In some cases, the patient has a serum bilirubin>3 mg/dL. Accordingly, one embodiment is the use of 25HC3S or salt thereof in treating or alleviating the symptoms of AH in a subject with serum bilirubin levels of >50 μmol/L, such as >60 μmol/L or >80 μmol/L, before administration of 25HC3S or salt thereof. In some cases, the patient to be treated has a bilirubin level ranging from about 2 mg/dL to 50 mg/dL, such as about 3 mg/dL to about 40 mg/dL or about 4 mg/dL to about 30 mg/dL. In certain cases, the patient to be treated is determined to have a bilirubin level that is >8 mg/dL.

In some embodiments, methods include determining serum bilirubin of the patient to be treated. The serum bilirubin of the patient can be determined using any convenient protocol, such as with a colorimetric assay or a fluorometric assay. In these embodiments, the serum bilirubin of the patient may be determined 10 minutes or more before the patient is treated according to the subject methods, such as 15 minutes or more, such as 30 minutes or more, such as 60 minutes or more, such as 2 hours or more, such as 6 hours or more and including 12 hours or more. In certain embodiments, the serum bilirubin of the patient is determined the day before the patient is to be treated according to the methods described herein.

In some instances, methods include determining serum bilirubin of the patient after one or more cycles of treatment according to the subject methods, such as 2 or more cycles, such as 3 or more cycles, such as 4 or more cycles and including 5 or more cycles. In these embodiments, the serum bilirubin may be determined immediately following the last administered dosage of 25HC3S or salt thereof or at a predetermined time thereafter, such as 5 minutes or more after the last administered dosage of 25HC3S or salt thereof, such as 10 minutes or more, such as 15 minutes or more, such as 30 minutes or more, such as 60 minutes or more, such as 2 hours or more, such as 6 hours or more and including 12 hours or more. In certain embodiments, the serum bilirubin of the patient is determined at some time on the same day as the last administered dosage of 25HC3S or salt thereof. In some cases, the patient has an abrupt rise in bilirubin, e.g, a rise of ≥3 mg/dL within 8 weeks. In some instances, the present methods are sufficient to reduce the serum bilirubin of the subject, such as by reducing serum bilirubin in the subject by 1 mg/dL or more, such as by 2 mg/dL or more and including by 3 mg/dL or more. In certain instances, where the subject exhibits an abrupt rise in bilirubin, the present methods are sufficient to reduce the rise in bilirubin by the amount elevated, such as by 1 mg/dL or more, such as by 2 mg/dL or more and including by 3 mg/dL or more. In some embodiments, the subject methods are sufficient to reduce serum bilirubin in the patient (e.g., at 7 days or 28 days after initiation of treatment) by 10% or more as compared to the serum bilirubin determined before treatment with 25HC3S or salt thereof as described herein, such as by 15% or more, such as by 20% or more, such as by 25% or more, such as by 30% or more, such as by 35% or more, such as by 40% or more, such as by 45% or more and including by 50% or more.

### MELD

The Model for End-Stage Liver Disease (MELD) is a scoring system for assessing the severity of liver disease. An increasing MELD score reflects greater risk of death, whereas a declining MELD score reflects a diminution in risk. MELD uses the patient's values for serum bilirubin, serum creatinine, and the international normalized ratio for prothrombin time (INR) to predict survival and is calculated according to the formula:

MELD=3.78×ln[serum bilirubin (mg/dL)]+11.2×ln[INR]+9.57×ln[serum creatinine (mg/dL)]+6.43

In some cases, the patient has a MELD score ranging from about 11 to about 40, such as about 11 to about 35, about 11 to about 33, or about 21 to about 30. In some cases, the patient has a MELD score≥11, such as ≥15 or ≥21. In some cases, the patient has a MELD score<35, such as <30, <25, <20, or <15. Accordingly, one embodiment is the use of 25HC3S or salt thereof in treating or alleviating the symptoms of AH in a subject, wherein the subject is characterized by a MELD score ranging from about 11 to about 30 or about 21 to about 30 before administration of 25HC3S or salt thereof.

In some embodiments, methods include determining the MELD score of the patient to be treated. In these embodiments, the MELD score of the patient may be determined 10 minutes or more before the patient is treated according to the subject methods, such as 15 minutes or more, such as 30 minutes or more, such as 60 minutes or more, such as 2 hours or more, such as 6 hours or more and including 12 hours or more. In certain embodiments, the MELD score of the patient is determined the day before the patient is to be treated according to the methods described herein.

In some instances, methods include determining the MELD score of the patient after one or more cycles of treatment according to the subject methods, such as 2 or more cycles, such as 3 or more cycles, such as 4 or more cycles and including 5 or more cycles. In these embodiments, the MELD score may be determined immediately following the last administered dosage of 25HC3S or salt thereof or at a predetermined time thereafter, such as 5 minutes or more after the last administered dosage of 25HC3S or salt thereof, such as 10 minutes or more, such as 15 minutes or more, such as 30 minutes or more, such as 60 minutes or more, such as 2 hours or more, such as 6 hours or more and including 12 hours or more. In certain embodiments, the MELD score of the patient is determined at some time on the same day as the last administered dosage of 25HC3S or salt thereof.

In some embodiments, the subject methods are sufficient to reduce the MELD score exhibited by the patient (e.g., at 7 days or 28 days after initiation of treatment) by 5% or more as compared to the MELD score determined before treatment with 25HC3S or salt thereof as described herein, such as by 10% or more, such as by 15% or more and including by 20% or more.

### MDF

The Maddrey discriminant function (MDF) is a model for evaluating the severity and prognosis in AH. The MDF score is a statistical model useful for predicting a subject's short term prognosis, in particular mortality within 30 or 90 days. A score of 32 or greater (severe alcoholic hepatitis (SAH)) implies poor outcome with 30-day mortality ranging from 35% to 45%. In contrast, MDF<32 identifies those with mild/moderate AH, conferring low, but not zero, risk of mortality with supportive care.

The MDF is calculated according to the formula:

$$M D F = 4.6 \times ( Prothrombin time ( {PT}_{PATIENT} - {PT}_{CONTROL} ) + Serum Bilirubin ( μmol / L ) / 17.1$$

Accordingly, one embodiment is the use of 25HC3S or salt thereof in treating or alleviating the symptoms of AH in a subject, wherein the subject is characterized by an MDF score of ≥32 before administration of 25HC3S or salt thereof. Thus, in some cases, the patient has an MDF score≥32. In other cases, the patient has an MDF score<32.

In some embodiments, methods include determining the MDF score of the patient to be treated. In these embodiments, the MDF score of the patient may be determined 10 minutes or more before the patient is treated according to the subject methods, such as 15 minutes or more, such as 30 minutes or more, such as 60 minutes or more, such as 2 hours or more, such as 6 hours or more and including 12 hours or more. In certain embodiments, the MDF score of the patient is determined the day before the patient is to be treated according to the methods described herein.

In some instances, methods include determining the MDF score of the patient after one or more cycles of treatment according to the subject methods, such as 2 or more cycles, such as 3 or more cycles, such as 4 or more cycles and including 5 or more cycles. In these embodiments, the MDF score may be determined immediately following the last administered dosage of 25HC3S or salt thereof or at a predetermined time thereafter, such as 5 minutes or more after the last administered dosage of 25HC3S or salt thereof, such as 10 minutes or more, such as 15 minutes or more, such as 30 minutes or more, such as 60 minutes or more, such as 2 hours or more, such as 6 hours or more and including 12 hours or more. In certain embodiments, the MDF score of the patient is determined at some time on the same day as the last administered dosage of 25HC3S or salt thereof.

In some embodiments, the subject methods are sufficient to reduce the MDF score exhibited by the patient (e.g., at 7 days or 28 days after initiation of treatment) by 5% or more as compared to the MDF score determined before treatment with 25HC3S or salt thereof as described herein, such as by 10% or more, such as by 15% or more and including by 20% or more.

In one embodiment provided is the use of 25HC3S or salt thereof in treating or alleviating the symptoms of AH in a subject, wherein the subject is characterized by an MDF score≥32 and a MELD score of <25 before administration of 25HC3S or salt thereof. In some instances, the present methods are sufficient to reduce the MDF score of a subject by 1 or more, such as 2 or more and including by 5 or more. In certain instances, the present methods are sufficient to reduce the MDF score of a subject having a score≥32 to a MDF score that is <32.

### ABIC

While the MDF is predictive at 1 month, it is less accurate in the intermediate and long-term. The MELD score and the more recently validated Age, serum Bilirubin, INR, and serum Creatinine (ABIC) score provide more nuanced survival prediction by emphasizing impaired renal function and can be calculated at different time points. An ABIC score of >9 indicates a high mortality risk, 6.71 to 9 indicates moderate mortality risk, and <6.71 indicates low mortality risk. Accordingly, in some cases the patient has an ABIC score of >9 or 6.71 to 9.

In some embodiments, methods include determining the ABIC score of the patient to be treated. In these embodiments, the ABIC score of the patient may be determined 10 minutes or more before the patient is treated according to the subject methods, such as 15 minutes or more, such as 30 minutes or more, such as 60 minutes or more, such as 2 hours or more, such as 6 hours or more and including 12 hours or more. In certain embodiments, the ABIC score of the patient is determined the day before the patient is to be treated according to the methods described herein.

In some instances, methods include determining the ABIC score of the patient after one or more cycles of treatment according to the subject methods, such as 2 or more cycles, such as 3 or more cycles, such as 4 or more cycles and including 5 or more cycles. In these embodiments, the ABIC score may be determined immediately following the last administered dosage of 25HC3S or salt thereof or at a predetermined time thereafter, such as 5 minutes or more after the last administered dosage of 25HC3S or salt thereof, such as 10 minutes or more, such as 15 minutes or more, such as 30 minutes or more, such as 60 minutes or more, such as 2 hours or more, such as 6 hours or more and including 12 hours or more. In certain embodiments, the ABIC score of the patient is determined at some time on the same day as the last administered dosage of 25HC3S or salt thereof.

In some embodiments, the subject methods are sufficient to reduce the ABIC score exhibited by the patient (e.g., at 7 days or 28 days after initiation of treatment) by 5% or more as compared to the ABIC score determined before treatment with 25HC3S or salt thereof as described herein, such as by 10% or more, such as by 15% or more and including by 20% or more.

### Glasgow AH Score

The Glasgow Alcoholic Hepatitis Score (GAHS) can be used to identify patients at risk of mortality (Forrest et al., Gut, 56:1743-1746 (2007)). A score of 9 or more identify patients most at risk of death.

In some embodiments, methods include determining the GAHS of the patient to be treated. In these embodiments, the GAHS of the patient may be determined 10 minutes or more before the patient is treated according to the subject methods, such as 15 minutes or more, such as 30 minutes or more, such as 60 minutes or more, such as 2 hours or more, such as 6 hours or more and including 12 hours or more. In certain embodiments, the GAHS of the patient is determined the day before the patient is to be treated according to the methods described herein.

In some instances, methods include determining the GAHS of the patient after one or more cycles of treatment according to the subject methods, such as 2 or more cycles, such as 3 or more cycles, such as 4 or more cycles and including 5 or more cycles. In these embodiments, the GAHS may be determined immediately following the last administered dosage of 25HC3S or salt thereof or at a predetermined time thereafter, such as 5 minutes or more after the last administered dosage of 25HC3S or salt thereof, such as 10 minutes or more, such as 15 minutes or more, such as 30 minutes or more, such as 60 minutes or more, such as 2 hours or more, such as 6 hours or more and including 12 hours or more. In certain embodiments, the GAHS of the patient is determined at some time on the same day as the last administered dosage of 25HC3S or salt thereof.

In some embodiments, the subject methods are sufficient to reduce the GAHS exhibited by the patient (e.g., at 7 days or 28 days after initiation of treatment) by 5% or more as compared to the GAHS determined before treatment with 25HC3S or salt thereof as described herein, such as by 10% or more, such as by 15% or more and including by 20% or more.

Accordingly, it is one aspect of the disclosure to improve the GAHS score. One embodiment is the use of 25HC3S or salt thereof in treating or alleviating the symptoms of AH in a subject, wherein the subject is characterized by a lowered GAHS score at least 7 days, at least 14 days, at least 21 days, at least 28 days, at least 42 days or at least 90 days compared to before first administration of 25HC3S or salt thereof. Another embodiment is the use of 25HC3S or salt thereof in treating or alleviating the symptoms of AH in a subject, wherein the subject is characterized by a lowered GAHS score at least 7 days, at least 14 days, at least 21 days, at least 28 days, at least 42 days or at least 90 days compared to before first administration of 25HC3S or salt thereof.

### Lille

The Lille score predicts mortality in patients with AH. The Lille score is calculated according to the following formula:

$$Exp ( - R ) / [ 1 + exp ( - R ) ]$$

$$where$$

$$R = [ 3.19 - ( 0.101 * age in years ) ] + ( 1.47 * albumin at baseline in g / dL ) + \text{} [ \text{} 0.28215 * ( bilirubin at baseline - bilirubin at Day 8 in mg / dL ) ] - [ \text{} 0.206 * ( if creatinine >= 1.3 mg / dL at baseline ) ] - \text{} [ \text{} 0.11115 * bilirubin baseline in mg / dL ] - ( 0.0096 * Prothrombin Time in seconds at baseline )$$

Accordingly, it is one aspect of the disclosure to improve the Lille score. One embodiment is the use of 25HC3S or salt thereof in treating or alleviating the symptoms of AH in a subject and wherein the subject is characterized by a lowered Lille score at 7 days after first administration of 25HC3S or salt thereof. In yet another embodiment provided is the use of 25HC3S or salt thereof in treating or alleviating the symptoms of AH in a subject, comprising administering 25HC3S or salt thereof, and wherein the subject is characterized by lowered Lille score at 7 days after first administration of 25HC3S or salt thereof. In some cases, the patient has a Lille score of <0.45 at 7 days after first administration of 25HC3S or salt thereof.

$$M E L D + Lille$$

Combining static and dynamic models to enhance prediction in AH, Louvet et al. demonstrated that a joint-effect model of MELD plus Lille outperformed other combinations such that for a patient with MELD 21 and Lille 0.45 had a 1.9-fold higher risk of death at 2 months than one with MELD 21 and Lille 0.16 (23.7% vs 12.5%). Thus, in some cases, the subject has been diagnosed with AH by use of a joint-effect model of MELD plus Lille.

In some cases, the subject has been diagnosed with AH by use of the alcoholic hepatitis histologic score (AHHS). The AAHS is based on four histologic parameters: degree of fibrosis, degree of neutrophil infiltration, type of bilirubinostasis, and presence of megamitochondria.

In some cases, the patient has aspartate aminotransferase (AST)>alanine transaminase (ALT), but less than 300 IU/L. In some cases, the patient has AST>50, AST/ALT>1.5, and both values<400 IU/L.

In certain embodiments, the subject has aspartate aminotransferase (AST) of from 50 to 400 IU/L. In certain embodiments, the subject has alanine transaminase (ALT) of less than 400 IU/L, such as 390 IU/L or less, such as 380 IU/L or less, such as 370 IU/L or less, such as 360 IU/L and including 350 IU/L. In certain embodiments, the subject has AST greater than ALT, such as where the ratio of AST to ALT (i.e., AST/ALT) is 1.05 or more, such as 1.1 or more, such as 1.15 or more, such as 1.2 or more, such as 1.25 or more, such as 1.3 or more, such as 1.35 or more, such as 1.4 or more, such as 1.45 or more and including 1.5 or more (e.g., preferably wherein AST/ALT of greater than 1.5). For instance, in certain embodiments, the subject may have a combination of two or more of these features, such as AST of from 50 to 400 IU/L, ALT of less than 400 IU/L, and AST/ALT of greater than 1.5. Furthermore, the patient may still further have one or more additional clinical features, for instance onset of jaundice within prior 8 weeks, serum total bilirubin>3.0 mg/dL, Maddrey's discriminant function≥32 assuming a control prothrombin time of 12 seconds, and/or MELD score of 21-30.

In some cases, the patient has an albumin level ranging from about 1 g/dL to about 5 g/dL, such as about 1 g/dL to about 4 g/dL or about 2 g/dL to about 3 g/dL.

In some cases, the patient has an international normalized ratio for prothrombin time (INR) ranging from about 10 seconds to about 30 seconds, such as about 11 seconds to about 25 seconds or about 15 seconds to about 25 seconds. In some embodiments, methods include determining the international normalized ratio for prothrombin time (INR) of the patient to be treated. In these embodiments, the international normalized ratio for prothrombin time of the patient may be determined 10 minutes or more before the patient is treated according to the subject methods, such as 15 minutes or more, such as 30 minutes or more, such as 60 minutes or more, such as 2 hours or more, such as 6 hours or more and including 12 hours or more. In certain embodiments, the international normalized ratio for prothrombin time of the patient is determined the day before the patient is to be treated according to the methods described herein.

In some instances, methods include determining the international normalized ratio for prothrombin time of the patient after one or more cycles of treatment according to the subject methods, such as 2 or more cycles, such as 3 or more cycles, such as 4 or more cycles and including 5 or more cycles. In these embodiments, the international normalized ratio for prothrombin time may be determined immediately following the last administered dosage of 25HC3S or salt thereof or at a predetermined time thereafter, such as 5 minutes or more after the last administered dosage of 25HC3S or salt thereof, such as 10 minutes or more, such as 15 minutes or more, such as 30 minutes or more, such as 60 minutes or more, such as 2 hours or more, such as 6 hours or more and including 12 hours or more. In certain embodiments, the international normalized ratio for prothrombin time of the patient is determined at some time on the same day as the last administered dosage of 25HC3S or salt thereof.

In some embodiments, the subject methods are sufficient to reduce the international normalized ratio for prothrombin time exhibited by the patient (e.g., at 7 days or 28 days after initiation of treatment) by 5% or more as compared to the international normalized ratio for prothrombin time determined before treatment with 25HC3S or salt thereof as described herein, such as by 10% or more, such as by 15% or more and including by 20% or more.

In some cases, the patient has a serum creatinine level ranging from about 0.2 mg/dL to about 3 mg/dL, such as about 0.4 mg/dL to about 2.5 mg/dL or about 0.6 mg/dL to about 2 mg/dL. In some embodiments, methods include determining the serum creatinine level of the patient to be treated. In these embodiments, the serum creatinine level of the patient may be determined 10 minutes or more before the patient is treated according to the subject methods, such as 15 minutes or more, such as 30 minutes or more, such as 60 minutes or more, such as 2 hours or more, such as 6 hours or more and including 12 hours or more. In certain embodiments, the serum creatinine level of the patient is determined the day before the patient is to be treated according to the methods described herein.

In some instances, methods include determining the serum creatinine level of the patient after one or more cycles of treatment according to the subject methods, such as 2 or more cycles, such as 3 or more cycles, such as 4 or more cycles and including 5 or more cycles. In these embodiments, the serum creatinine level may be determined immediately following the last administered dosage of 25HC3S or salt thereof or at a predetermined time thereafter, such as 5 minutes or more after the last administered dosage of 25HC3S or salt thereof, such as 10 minutes or more, such as 15 minutes or more, such as 30 minutes or more, such as 60 minutes or more, such as 2 hours or more, such as 6 hours or more and including 12 hours or more. In certain embodiments, the serum creatinine level of the patient is determined at some time on the same day as the last administered dosage of 25HC3S or salt thereof.

In some embodiments, the subject methods are sufficient to reduce the serum creatinine level of the patient (e.g., at 7 days or 28 days after initiation of treatment) by 2% or more as compared to the serum creatinine level determined before treatment with 25HC3S or salt thereof as described herein, such as by 5% or more, such as by 10% or more and including by 15% or more.

In some cases, the patient has acute AH presenting as acute on chronic liver failure (ACLF).

In some cases, the subject has been diagnosed with AH based on circulating fragments of cytokeratin-18 (CK-18) and the main constituent of Mallory-Denk bodies, termed M65 and M30.

In view of the above, the diagnosis of AH (i.e., the identification of subject experiencing AH and who could benefit from the practice of the present methods) is generally based on physical exam, laboratory findings, patient history, and past medical history to establish, for example, mental status changes, coagulopathy, rapidity of onset, and absence of known prior liver disease. In some cases, the AH patient may have other prior liver disease, such as cholestatic liver disorder, autoimmune liver disorder, active viral hepatitis, Wilson's disease, or other liver disorders not caused by alcohol consumption.

The definition of “rapid” depends on the particular convention that is used. Different sub-divisions exist which are based on the time from onset of first hepatic symptoms to onset of encephalopathy. In some cases, “AH” involves the development of encephalopathy, jaundice, or rise of bilirubin to >3 mg/dl within 26 weeks of the onset of any hepatic symptoms. This is sub-divided into “fulminant hepatic failure,” which requires onset of encephalopathy, jaundice, or rise of bilirubin to >3 mg/dL within 8 weeks, and “subfulminant,” which describes onset of encephalopathy, jaundice, or rise of bilirubin to >3 mg/dL after 8 weeks but before 26 weeks from hepatic symptoms. In other cases, “hyperacute” liver failure may be characterized as onset within 7 days from hepatic symptoms, “acute” liver failure as onset between 7 and 28 days from hepatic symptoms, and “subacute” liver failure as onset between 28 days and 24 weeks from hepatic symptoms. Subjects identified as experiencing acute liver failure by any of these criteria may be treated by the methods described herein.

In some aspects, the populations of subjects treated by the methods described herein may or may not have symptoms of and/or been diagnosed with high levels of cholesterol (hypercholesterolemia, e.g., cholesterol levels in serum in the range of about 200 mg/dl or more), or with a condition associated with high levels of cholesterol, e.g., hyperlipidemia, atherosclerosis, heart disease, stroke, Alzheimer's, gallstone diseases, cholestatic liver diseases, etc. In some aspects, the populations of subjects treated by the methods described herein do not have symptoms of and/or have not been diagnosed with high levels of cholesterol (hypercholesterolemia, e.g., cholesterol levels in serum in the range of about 200 mg/dl or more), or with a condition associated with high levels of cholesterol, e.g., hyperlipidemia, atherosclerosis, heart disease, stroke, Alzheimer's, gallstone diseases, cholestatic liver diseases, etc.

### Description of Administration

Implementation of the methods generally involves identifying patients suffering from AH and administering 25HC3S or salt thereof in an acceptable form by an appropriate route. The exact total amount to be administered (e.g., in a period of one month) may vary depending on the age, gender, weight and overall health status of the individual patient, as well as the precise etiology of the disease.

In some embodiments, the total amount of 25HC3S or salt thereof administered to the patient is surprisingly low. The total amount administered (e.g., in a period of one month) typically ranges from about 0.01 mg/kg to about 50 mg/kg, more usually from about 0.05 mg/kg to about 20 mg/kg, such as from about 0.1 mg/kg to about 10 mg/kg, from about 0.2 mg/kg to about 6 mg/kg, from about 0.2 mg/kg to about 2 mg/kg, or from about 0.3 mg/kg to about 1.5 mg/kg, of 25HC3S or salt thereof per kg of body weight.

Total amounts generally range from about 1 mg to about 1000 mg of 25HC3S or a salt thereof. In some embodiments, the total amount of 25HC3S or salt thereof administered to the patient in the methods (e.g., in a period of one month) is from about 10 mg to about 500 mg. Typically, in some embodiments, the total amount of 25HC3S or salt thereof administered to the patient in the methods (e.g., in a period of one month) is from about 10 mg to about 400 mg. For instance, in some embodiments, the total amount is at least about 20 mg. In some embodiments, the total amount is not more than about 300 mg, such as not more than about 200 mg, or even not more than about 140 mg. Particular examples of total amounts administered include from about 10 mg to about 500 mg, from about 10 mg to about 400 mg, from about 20 mg to about 300 mg, from about 20 mg to about 200 mg, and from about 20 mg to about 140 mg. The total amount will vary with the route of administration, the bioavailability, and the particular formulation that is administered. For instance, for intravenous administration, the total amounts described herein may be particularly useful.

In some embodiments, the low number of doses is surprising (including, but not limited to, when the 25HC3S or salt thereof is administered by intravenous administration). In this regard, the total amount of 25HC3S or salt thereof to be administered in the methods can be administered in one dose or in a plurality of separate doses over a period of time (such as a period of one month). In some embodiments, the total amount is administered in from one to fifteen separate doses, e.g., in from one to five separate doses, such as in one dose or in two separate doses. In some embodiments, the total amount is administered in a single dose. In other embodiments, the total amount is administered in two separate doses. In all methods described herein, the total amount administered comprises at least a first dose (this first dose being the single dose when the total amount is administered in a single dose, but being the first dose followed by at least one further dose in other embodiments).

In some embodiments, at least one, and preferably each, of the one or more separate doses comprises from about 10 mg to about 200 mg of 25HC3S or salt thereof. For instance, in some embodiments at least one, and preferably each, of the one or more separate doses comprises from about 10 mg to about 120 mg of 25HC3S or salt thereof, such as from about 20 mg to about 100 mg of 25HC3S or salt thereof. Specific exemplary dose amounts include doses of: (a) from about 20 mg to about 40 mg of 25HC3S or salt thereof (such as about 30 mg): or (b) from about 80 mg to about 100 mg of 25HC3S or salt thereof (such as about 90 mg): or (c) from about 140 mg to about 160 mg of 25HC3S or salt thereof (such as about 150 mg). In some embodiments, particularly useful dose amounts may be (a) or (b) above. In some embodiments, particular useful numbers of separate doses for such dose amounts may be one dose (i.e., a single dose) or two separate doses.

In some embodiments, the low frequency of dosing is surprising. For example, in some embodiments the total amount is administered in two or more separate doses that are administered at a dose frequency ranging from daily to once every week, such as from once every two days to once every week, e.g., from once every two days to once every five days or once every two days to once every four days. An example of such a dose frequency is administration once about every three days. For instance, when the total number of separate doses is two, the second dose may be administered after the first dose by from one day to one week, by from two days to one week, by from two days to five days, by from two days to four days, or by about three days.

In some embodiments, the one or more separate doses are administered over an administration period (meaning the period in which actual administration of the 25HC3S or salt thereof takes place). For instance, when the method comprises administering the total amount in a period of one month measured from the beginning of administration of the first dose, then the administration period can be less than the period of one month. Thus, the administration period is in some embodiments followed by a “non-administration period” during which time there is no administration of 25HC3S or salt thereof, such that the administration period and non-administration period add up in total to the period of one month. In some such embodiments, the administration is not more than 21 days, such as not more than 14 days, e.g., not more than 7 days or not more than 5 days.

In view of the above, the total amount of 25HC3S or salt thereof administered in a period of one month may range from about 0.01 mg/kg/month to about 50 mg/kg/month, more usually from about 0.05 mg/kg/month to about 20 mg/kg/month, such as from about 0.1 mg/kg/month to about 10 mg/kg/month, e.g., from about 0.2 mg/kg/month to about 6 mg/kg/month or from about 0.3 mg/kg/month to about 1.5 mg/kg/month. The total amount of 25HC3S or salt thereof administered in a period of one month may range from about 1 mg/month to about 1000 mg/month, from about 5 mg/month to about 600 mg/month, from about 10 mg/month to about 400 mg/month, from about 20 mg/month to about 300 mg/month, from about 20 mg/month to about 200 mg/month, or from about 20 mg/month to about 140 mg/month.

The administration of the compound of the present disclosure may be intermittent, or at a gradual or continuous, constant or controlled rate.

Administration may be through any route, such as parenteral, including injection intravenously, intramuscularly, and/or subcutaneously. The route of administration will depend on the nature of the condition that is treated, e.g., on the type or degree of liver injury and/or liver failure. For example, to achieve expedited treatment before significant liver dysfunction or failure has occurred, dosing by intravenous injection may be preferred. Thus, when damage has already occurred, and especially when acute organ failure is diagnosed, the route of administration is generally parenteral or intravenous to speed delivery of the 25HC3S or salt thereof.

The 25HC3S may be administered in the pure form or in a pharmaceutically acceptable formulation including suitable elixirs and the like (generally referred to a “carriers”) or as pharmaceutically acceptable salts (e.g., alkali metal salts such as sodium, potassium, calcium or lithium salts, ammonium, etc.) or other complexes. It may, for instance, be preferable to utilize a salt of 25HC3S: the sodium salt of 25HC3S is one exemplary such salt. It should be understood that the pharmaceutically acceptable formulations include liquid materials conventionally utilized to prepare injectable dosage forms. The 25HC3S or salt thereof is typically administered as compositions that are liquids suitable for injection and/or intravenous administration. Solid forms suitable for solution in, or suspension in, liquids prior to administration may also be prepared.

The active ingredients may be mixed with excipients which are pharmaceutically acceptable and compatible with the active ingredients, e.g., pharmaceutically and physiologically acceptable carriers. Suitable excipients include, for example, water, saline (sodium chloride), cyclodextrin (e.g., hydroxypropyl-beta-cyclodextrin), dextrose, glycerol, ethanol and the like, or combinations thereof. In addition, the composition may contain minor amounts of auxiliary substances such as wetting or emulsifying agents, pH buffering agents (e.g., phosphate buffer), and the like. Water may be used as the carrier for the preparation of compositions (e.g., injectable compositions), which may also include conventional buffers and agents to render the composition isotonic. Other potential additives and other materials (preferably those which are generally regarded as safe [GRAS]) include: surfactants (TWEEN®, oleic acid, etc.); solvents, stabilizers, elixirs, and encapsulants (lactose, liposomes, etc). Preservatives such as methyl paraben or benzalkium chloride may also be used. The composition of the present disclosure may contain any such additional ingredients so as to provide the composition in a form suitable for the intended route of administration. In addition, the compounds may be formulated with aqueous or oil based vehicles.

Depending on the formulation, it is expected that the 25HC3S or salt thereof will be present at about 1 wt % to about 99 wt % of the composition and the vehicular “carrier” will constitute about 1 wt % to about 99 wt % of the composition. The pharmaceutical compositions of the present disclosure may include any suitable pharmaceutically acceptable additives or adjuncts to the extent that they do not hinder or interfere with the therapeutic effect of the 25HC3S or salt thereof.

In some cases, the alcoholic hepatitis is characterized by reduced liver function such that the subject exhibits a reduced rate of clearance of 25HC3S as compared to a subject that does not have alcoholic hepatitis. In some cases, the alcoholic hepatitis is characterized by reduced liver function such that the subject exhibits a rate of clearance of 25HC3S that is 50% or less, 40% or less, 30% or less, or 25% or less of the rate of clearance of 25HC3S by a subject that does not have alcoholic hepatitis. In one exemplary, but non-limiting, case, the rate of clearance of 25HC3S by a subject that does not have alcoholic hepatitis, against which reduced rate of clearance in the subject having alcoholic hepatitis is measured, is considered to be 25 L/hr (i.e., the present subject exhibits a rate of clearance of 25HC3S of less than 25 L/hr, e.g. 50% or less, 40% or less, 30% or less, or 25% or less than 25 L/hr).

In some cases, the alcoholic hepatitis is characterized by reduced liver function such that the subject exhibits a half-life time of 25HC3S in the plasma after administration (T_{1/2}) that is 1.5-fold greater or more, or 2-fold greater or more, as compared to a subject that does not have alcoholic hepatitis. In one exemplary, but non-limiting, case, the half-life time of a subject that does not have alcoholic hepatitis, against which greater half-life time in the subject having alcoholic hepatitis is measured, is considered to be 1.6 hr (i.e., the present subject exhibits a half-life time that is greater than 1.6 hr such as 1.5-fold greater or more, or 2-fold greater or more). In some cases, the time to maximum drug concentrations (Tₘₐₓ) is at the end of infusion, e.g., 2-hour infusion. In some embodiments, the half-life (t_{1/2}) of 25HC3S or salt thereof ranges from about 4 to about 6 hours. In some cases, the subject exhibits a half-life time of 25HC3S after administration (T_{1/2}) ranging from about 1.5 hours to about 6 hours or from about 2 hours to about 5 hours.

In some cases, mean clearance of 25HC3S or salt thereof is about 5 L/hr to about 7 L/hr. In some cases, the subject exhibits a clearance of 25HC3S ranging from about 2 L to about 8 L/h, about 2.5 L/h to about 7.5 L/h, or from about 3 L/h to about 7 L/h.

In some cases, the subject exhibits a volume of distribution of 25HC3S ranging from about 10 L to about 50 L, about 15 L to about 45 L, or from about 20 L to about 40 L. In some cases, the subject exhibits a volume of distribution that is lower than the volume of distribution of 25HC3S exhibited by a subject that does not have alcoholic hepatitis (which, may, for instance, be considered to be 55 L)—e.g. 90% or less, 80% or less, 70% or less, or 60% or less).

In some cases, the subject exhibits a Cmax of 25HC3S ranging from about 500 ng/mL to about 10,000 ng/ml, from about 600 ng/ml to about 7000 ng/ml, or from about 700 ng/mL to about 5000 ng/mL. For instance, in some cases, the subject exhibits a Cmax of 25HC3S ranging from about 500 ng/ml to about 10,000 ng/ml, from about 600 ng/ml to about 7000 ng/ml or from about 700 ng/ml to about 5000 ng/ml, per 100 mg of intravenously administered 25HC3S or salt thereof.

In some cases, the subject exhibits an AUCinf of 25HC3S ranging from about 3000 ng*h/mL to about 50,000 ng*h/mL, about 4000 ng*h/mL to about 40,000 ng*h/mL or from about 5000 ng*h/mL to about 30,000 ng*h/mL. For instance, in some cases, the subject exhibits an AUCinf of 25HC3S ranging from about 5000 ng*h/mL to about 30,000 ng*h/mL or from about 6000 ng*h/mL to about 20,000 ng*h/mL, per 100 mg of intravenously administered 25HC3S or salt thereof.

In one embodiment, 25HC3S or salt thereof is used for treating or alleviating the symptoms of AH in a subject, wherein the subject has reduced risk of mortality 90 days after first administration of 25HC3S or salt thereof compared to a subject not receiving 25HC3S or salt thereof. In certain embodiments, the risk of mortality 90 days after first administration of 25HC3S or salt thereof compared to a subject not receiving 25HC3S or salt thereof is reduced by 5% or more, such as by 10% or more, such as by 25% or more, such as by 50% or more, such as by 75% or more, such as by 90% or more and including where the subject has reduced risk of mortality 90 days after first administration of 25HC3S or salt thereof 99% or more as compared to a subject not receiving 25HC3S or salt thereof.

In one embodiment, 25HC3S or salt thereof is used for treating or alleviating the symptoms of AH in a subject, wherein the subject has reduced risk of mortality 28 days after first administration of 25HC3S or salt thereof compared to a subject not receiving 25HC3S or salt thereof. In certain embodiments, the risk of mortality 28 days after first administration of 25HC3S or salt thereof compared to a subject not receiving 25HC3S or salt thereof is reduced by 5% or more, such as by 10% or more, such as by 25% or more, such as by 50% or more, such as by 75% or more, such as by 90% or more and including where the subject has reduced risk of mortality 90 days after first administration of 25HC3S or salt thereof 99% or more as compared to a subject not receiving 25HC3S or salt thereof.

In certain embodiments, methods and compositions of the present disclosure are sufficient to reduce the duration of hospitalization as compared to a subject not receiving 25HC3S or salt thereof. In some instances, the subject methods and compositions are sufficient to reduce hospitalization, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the duration of hospitalization by 40% or more.

In certain embodiments, methods and compositions of the present disclosure are sufficient to reduce the duration of intensive care unit treatment as compared to a subject not receiving 25HC3S or salt thereof. In some instances, the subject methods and compositions are sufficient to reduce duration of intensive care unit treatment, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the duration of intensive care unit treatment by 40% or more.

The present invention will be further illustrated by way of the following Examples. These Examples are non-limiting and do not restrict the scope of the invention. Unless stated otherwise, all percentages, parts, etc. presented in the Examples are by weight.

### EXAMPLES

### Example 1

As described in more detail below, a total of 19 patients were enrolled into an open-label Phase 2a multi-center trial, of whom 15 had Maddrey's Discriminant Function (MDF)≥32 (Severe Alcoholic Hepatitis (SAH)), 12 had MELD scores at 21-30, and 11 had baseline serum bilirubin levels>8 mg/dL. All patients received IV infused 25HC3S (at 30, 90, or 150 mg) on Day 1 and Day 4 (if still hospitalized), and were followed for 28-days. The final enrollment was 19 patients comprised of: 8 patients (4 moderate and 4 severe) dosed at 30 mg, 7 patients (3 moderate and 4 severe) dosed at 90 mg, and 4 patients (4 severe) dosed at 150 mg.

### Methods

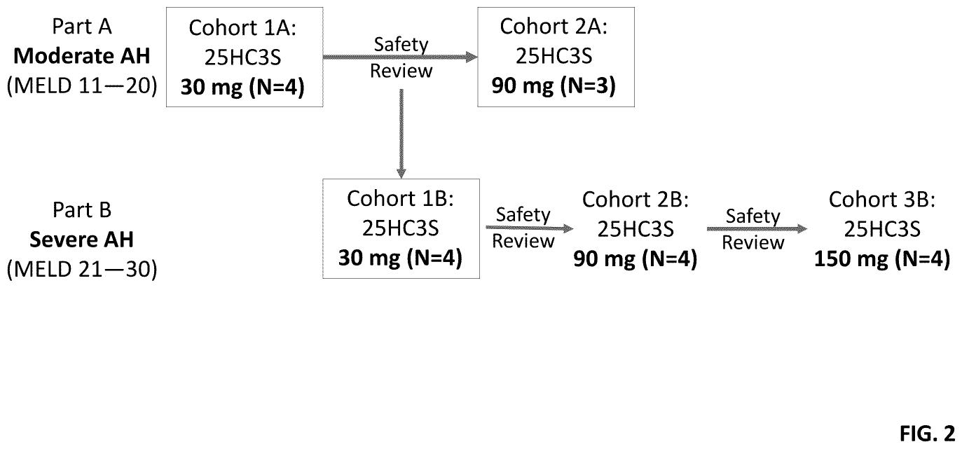

Study Drug A 25HC3S sodium salt solution contained:
   30 mg/mL 25HC3S sodium salt;
   240 mg/mL hydroxypropyl-beta-cyclodextrin; and
   50 mmole phosphate buffer
The 25HC3S sodium salt solution was diluted in 100 mL 5% dextrose or 0.9% sodium chloride infusion solution:
   Dose 1:30 mg of 25HC3S sodium salt in 100 mL 5% dextrose or 0.9% sodium chloride
   Dose 2:90 mg of 25HC3S sodium salt in 100 mL 5% dextrose or 0.9% sodium chloride
   Dose 3:150 mg of 25HC3S sodium salt in 100 mL 5% dextrose or 0.9% sodium chloride
25HC3S sodium salt was administered by slow intravenous infusion over 2 hrs (50 mL/h) until the entire dose is given at Day 1 and Day 4. If a patient met the hospital discharge criteria prior to the second dose, the patient received only one dose instead of two doses.
Study Procedure The study was conducted in two parts. Part A included patients with MELD scores of 11-20 (moderate AH patients), and Part B included patients with MELD of scores 21-30. The doses of 25HC3S sodium salt administered were 30 mg and 90 mg for Part A and 30 mg, 90 mg and 150 mg for Part B, with 4 patients per dose group per Part (except for only 3 patients in the 90 mg cohort of Part A). All patients were followed to Day 28.
The study started in 4 moderate AH patients (Part A) at 30 mg dose level. After completing the dosing, a Dose Escalation Committee (DEC) reviewed safety, tolerability and pharmacokinetic (PK) data from all 4 patients and determined that it was safe to move forward to dose 4 severe AH patients (Part B) at the same dose level, 30 mg, as well as escalating the dose level to 90 mg in moderate patients (Part A). The same dose escalation procedure was then conducted in Part B until all 4 patients received 150 mg doses.
A flow diagram of the study is depicted in FIG. 2. Each dose cohort enrolled up to 4 subjects. Study subjects received up to 2 doses of 25HC3S sodium salt. The first dose was administered on Day 1 and, if still hospitalized, the second dose was administered on Day 4. A pharmacokinetic (PK) sample was collected at: Time 0 (pre-dose), 1 hour post-dose initiation, 2 hours (end of infusion), 3 hours post-dose initiation, 4 hours post-dose initiation, 8 hours post-dose initiation, 12 hours post-dose initiation, 24-hours post-dose initiation and 50 hours post-dose initiation.

### Population:

1. Patients with alcoholic hepatitis defined in this clinical trial as:
   a. History of heavy alcohol abuse: >40 g/day in females or >60 g/day in males for a minimum period of 6 months, AND
   b. Consumed alcohol within 12 weeks of entry into the study, AND
   c. Serum bilirubin>3 mg/dL AND AST>ALT, but less than 300 U/L, AND
   d. MELD score between 11-30, inclusive
      A listing of the baseline laboratory characteristics of the subjects of each cohort is shown in the Table below.

| | Study part | | | | | |
|---|---|---|---|---|---|---|
| | Part A: Moderate AH | | Part B: Severe AH | | | |
| | (MELD 11-20) | | (MELD 21-30) | | | |
| Study Cohort | 1A (30 mg) | 2A (90 mg) | 1B (30 mg) | 2B (90 mg) | 3B (150 mg) | Overall |
| (dosage) | N = 4 | N = 3 | N = 4 | N = 4 | N = 4 | (N = 19) |
| AST (IU/L) | 113.0 ± 112.0 | 112.7 ± 24.6 | 116.8 ± 30.1 | 89.5 ± 43.3 | 82.0 ± 18.6 | 102.3 ± 54.1 |
| ALT (IU/L) | 36.0 ± 38.1 | 67.3 ± 9.7 | 44.3 ± 15.4 | 30.5 ± 10.7 | 35.5 ± 20.3 | 41.4 ± 23.1 |
| T. Bilirubin | 5.5 ± 1.9 | 10.6 ± 5.7 | 18.7 ± 6.5 | 16.3 ± 10.5 | 19.1 ± 10.2 | 14.2 ± 8.7 |
| (mg/dL) | | | | | | |
| Creatinine | 0.63 ± 0.14 | 0.58 ± 0.34 | 0.86 ± 0.27 | 0.91 ± 0.43 | 0.68 ± 0.23 | 0.74 ± 0.29 |
| (mg/dL) | | | | | | |
| WBC (10³/μL) | 6.6 ± 8.0 | 9.9 ± 5.7 | 10.2 ± 3.2 | 6.2 ± 2.4 | 9.6 ± 4.7 | 8.4 ± 4.7 |
| Platelets | 113.8 ± 100.7 | 126.7 ± 7.8 | 179.0 ± 89.5 | 83.8 ± 32.5 | 173.0 ± 30.5 | 135.7 ± 69.4 |
| (K/μL) | | | | | | |
| INR | 1.7 ± 0.27 | 1.3 ± 0.22 | 1.8 ± 0.31 | 1.9 ± 0.29 | 2.1 ± 0.34 | 1.8 ± 0.35 |
| Maddrey's | 41.0 ± 12.2 | 25.7 ± 16.5 | 59.3 ± 18.5 | 63.3 ± 5.9 | 71.0 ± 20.0 | 53.4 ± 21.1 |
| Discriminant | | | | | | |
| Function | | | | | | |
| MELD | 18.5 ± 2.38 | 18.0 ± 3.61 | 24.5 ± 3.51 | 24.5 ± 2.89 | 25.0 ± 1.83 | 22.3 ± 4.06 |

Evaluation: Assessment of Pharmacodynamic (PD) Signals:
   Change in Lille score, MELD score, biochemistry (such as bilirubin)
   Method and Timing:
   Lille score was calculated at Day 7
   MELD score was calculated at Screening, Day 1 (pre-dose), Day 7 and Day 28
   Biochemistry and all safety lab parameters were collected at Screening, Day 1 and Day 4 at pre-dose, Day 1 and Day 4 at 12 hrs after dose completion/end of infusion, Day 2, Day 7, Day 28, and if hospitalized on Days 3, 4, 5, and 6.
   Assessment of Safety Signals:
   Safety was determined based on clinical and laboratory monitoring.
   Assessment of PK:
   Plasma concentration data of 25HC3S from each patient were used to calculate relevant PK parameters determined using a standard linear/log-trapezoidal rule non-compartmental method with an appropriate PK data analysis program.
   The time points for PK sample collection were (after Dose 1 only): 0 (pre-dose), 1 hr post-dose initiation, 2 hrs (end of infusion), 3, 4, 8, 12, 24 hrs post-dose

### Results

The 25HC3S at all 3 doses was safe with no drug-related serious adverse events. All patients, including SAH patients, survived the 28-day follow-up period. Some of the patients received one dose and some patients received two doses. The amount dosed ranged from 30 mg to 300 mg. Of the 18 patients that returned for the Day 7 visit, four received two doses and fourteen received one dose.

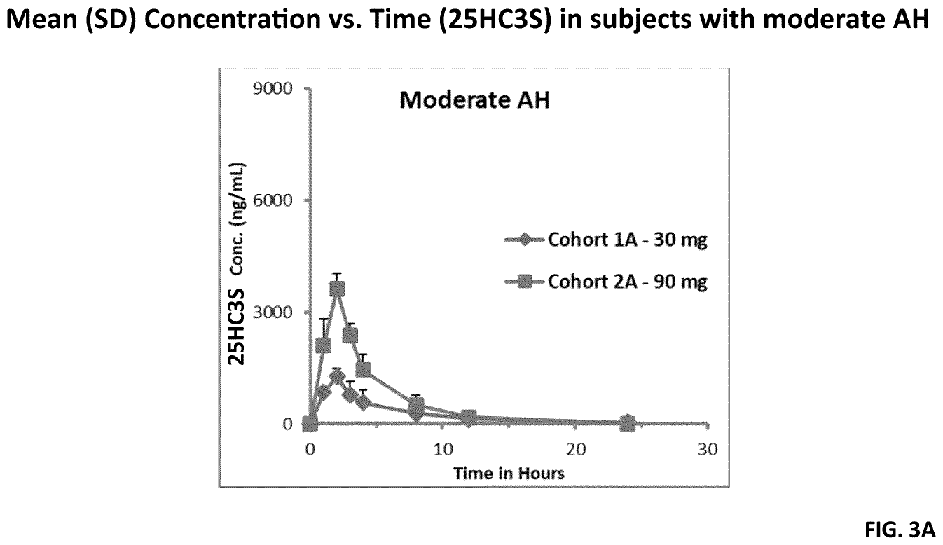

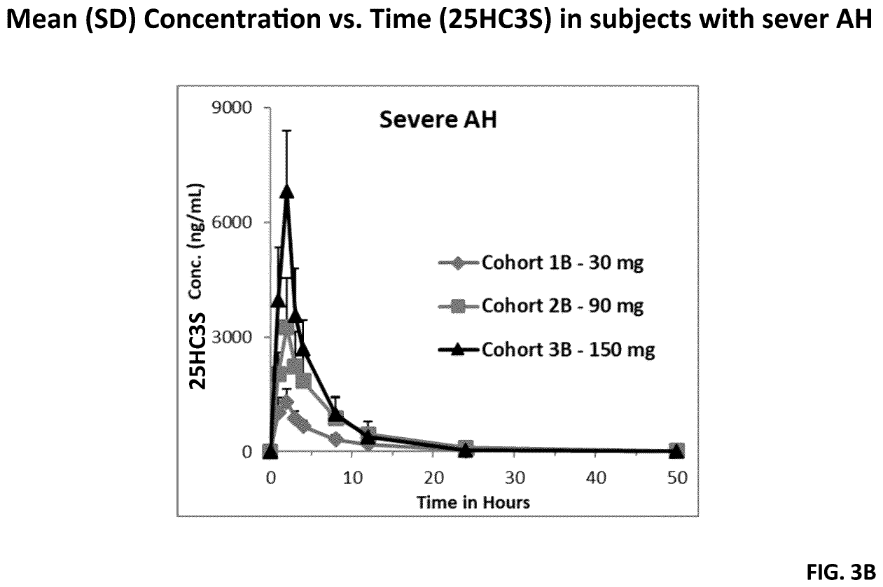

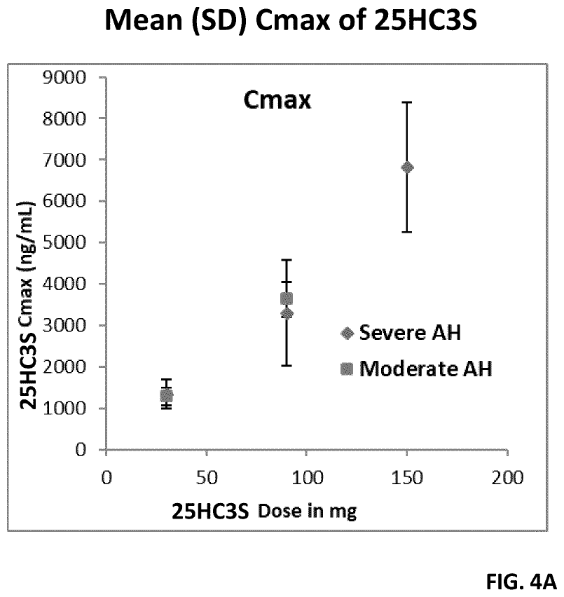

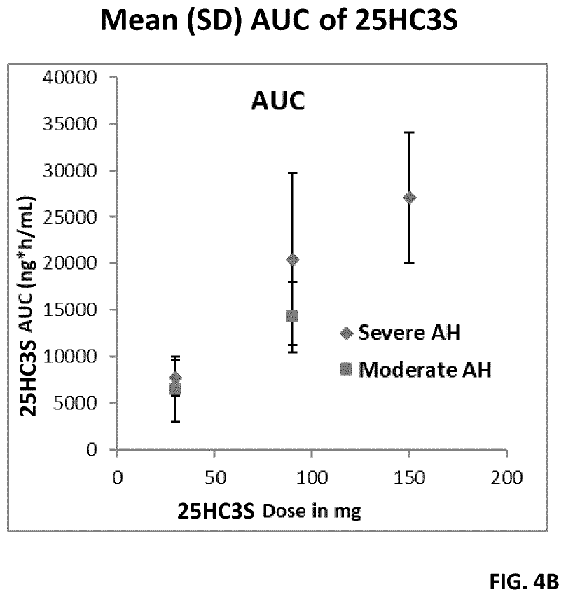

The drug exposure (both AUC and Cₘₐₓ) in AH patients was dose proportional. Time to maximum drug concentrations (Tₘₐₓ) was at the end of 2 hour infusion. The mean plasma concentration of 25HC3S sodium in subjects with moderate AH and severe AH is shown in FIG. 3A and FIG. 3B, respectively. The half-life (t_{1/2}) of 25HC3S sodium ranged from 4-6 hours. Mean clearance of 25HC3S sodium was about 5-7 L/hr. PK parameters of 25HC3S sodium were similar between the moderate and severe AH groups. A summary of the PK parameters in subjects with moderate AH and severe AH at each dosage level is summarized in the following Table. A plot of the Cmax (FIG. 4A) and AUC (FIG. 4B) for subjects with moderate AH and severe AH is depicted in FIG. 4.

| | | | | | | | |
|---|---|---|---|---|---|---|---|
| | | Tmax | Cmax | T½ | AUCinf | V | CL |
| Dose | N | (h) | (ng/mL) | (h) | (ng * h/mL) | (L) | (L/h) |

Moderate AH

| | | | | | | | |
|---|---|---|---|---|---|---|---|
| 1A (30 mg) | 3 | 2.2 (0.2) | 1277 (214) | 3.7 (1.8) | 6542 (3509) | 24.8 (1.6) | 5.9 (3.7) |
| 2A (90 mg) | 3 | 2.0 (0) | 3627 (418) | 2.7 (0.8) | 14264 (3759) | 25.8 (10.5) | 6.6 (1.5) |

Severe AH

| | | | | | | | |
|---|---|---|---|---|---|---|---|
| 1B (30 mg) | 4 | 1.8 (0.5) | 1343 (343) | 1.8 (0.5) | 7721 (1945) | 26.1 (4.9) | 4.1 (1.2) |
| 2B (90 mg) | 4 | 2.3 (0.5) | 3290 (1277) | 5.1 (1.9) | 20447 (9236) | 35.3 (10.8) | 5.5 (3.3) |
| 3B (150 mg) | 4 | 2.1 (0.3) | 6820 (1577) | 3.4 (0.4) | 27062 (6998) | 27.8 (5.9) | 5.8 (1.2) |

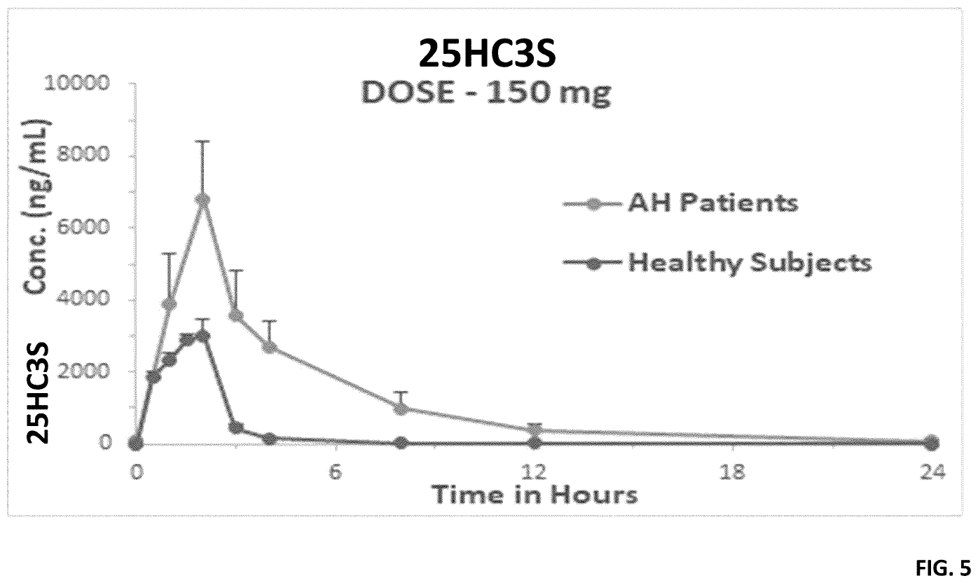

Compared to PK parameters of 25HC3S sodium in healthy subjects, there was a 2-fold increase in Cₘₐₓ and about 5-fold increase in AUC in AH patients at the same dose level (a single dose of 150 mg). The clearance of 25HC3S sodium was decreased by 80% in AH patients as compared to that in healthy subjects from an earlier study. A comparison of the pharmacokinetic parameters of 25HC3S administered to healthy subjects versus subjects with alcoholic hepatitis is summarized in the following Table. A comparison of the plasma concentration of 25HC3S administered to healthy subjects versus subjects with alcoholic hepatitis is depicted in FIG. 5.

| | Cmax | T½ | AUCinf | | |
|---|---|---|---|---|---|
| Subjects | (ng/mL) | (h) | (ng*h/mL) | V (L) | CL (L/h) |
| AH | 6820 | 3.4 (0.4) | 27062 (6998) | 27.8 (5.9) | 5.8 (1.2) |
| (N = 4) | (1577) | | | | |
| Healthy | 3102 | 1.6 (0.9) | 6192 (730) | 55.0 (4.3) | 24.5 (3.1) |
| (N = 5) | (294.2) | | | | |
| Fold | ↑ 2× | ↑ 2× | ↑ ~5× | ↓ 0.5× | ↓ 0.25× |
| Change | | | | | |
| in PK | | | | | |

Treatment responders (Lille score<0.45) among all 25HC3S treated AH patients (1 patient did not return for the Day 7 visit) were 89%; among 15 SAH patients (MDF≥32), 87%; and among 12 patients with MELD 21-30, 83%. In particular, 100% of SAH patients treated with 30 or 90 mg 25HC3S (n=11) responded to the treatment as shown in the following Table.

| | | Lille Median |
|---|---|---|
| AH Patient Category (n) | Responders | (1^{st}, 3^{rd} quartile) |
| All Patients (18) | 89% | 0.10 (0.04, 0.20) |
| with 30 or 90 mg 25HC3S (14) | 100% | 0.05 (0.04, 0.19) |
| SAH (15) | 87% | 0.19 (0.05, 0.22) |
| with 30 or 90 mg 25HC3S (11) | 100% | 0.12 (0.05, 0.19) |
| MELD 21-30 (12) | 83% | 0.19 (0.11, 0.25) |
| with 30 or 90 mg 25HC3S (8) | 100% | 0.19 (0.10, 0.19) |
| Baseline bilirubin >8 mg/dL (11) | 82% | 0.10 (0.05, 0.20) |
| with 30 or 90 mg 25HC3S (8) | 100% | 0.10 (0.05, 0.19) |

Although patients received only 1 or 2 doses of 25HC3S, their MELD scores on Day 28 in SAH patients were significantly reduced from baseline (−17.5%, p=0.01). The median reduction of MELD on Day 28 from baseline in SAH patients treated with 30 or 90 mg of 25HC3S was −19.0% (p=0.01). The 25HC3S also significantly reduced serum bilirubin levels on Day 7 in AH patients, especially in patients with baseline bilirubin>8.0 mg/dL (−25.1%, p=0.02).

Comparing our data to a set of data used for developing Lille score by Louvet et al., which evaluated 145 AH patients (with comparative DF scores, prothrombin times, and serum creatinine, albumin and bilirubin levels as those in this study), Lille scores from our study were significantly lower, p<0.0001, than that from the historical data, 0.24 (0.07, 0.60). Louvet et al., Hepatology, 45:1348-1354 (2007); and Louvet et al., Gastroenterology, 149:398-406 (2015).

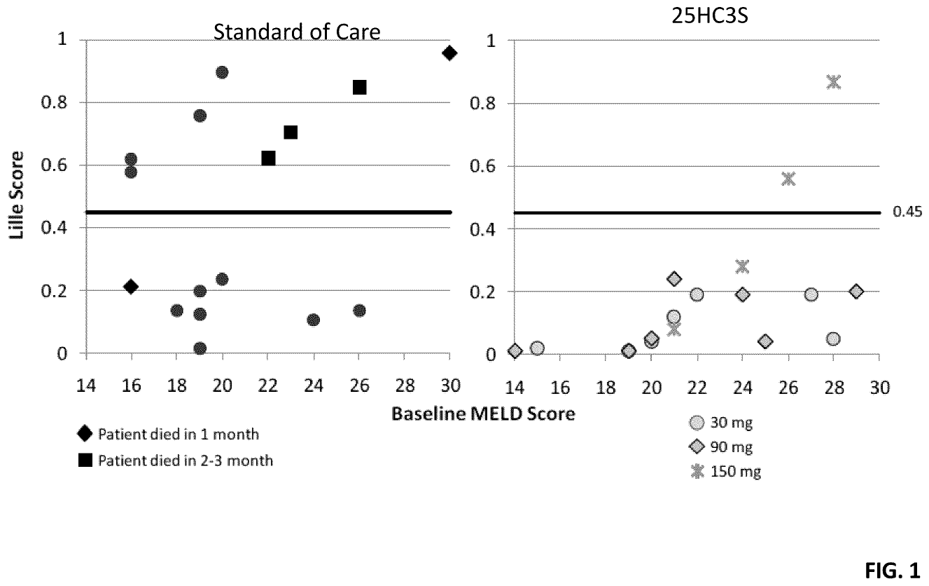

Clinical data from the trial also shows that the 19 patients treated with 25HC3S had statistically significantly greater reductions from baseline in bilirubin (day 7 and 28) and MELD (day 28), as well as statistically significantly lower Lille scores, compared with a historical control group (n=15) from a University of Louisville (UL) AH study. See FIG. 1. The UL shared anonymized data in which 16 AH patients with initial MELD scores ranging from 15-30 received either supportive care alone (n=9) or supportive care with corticosteroids (n=7). Of the 19 AH patients with 25HC3S, one patient did not return for the day 7 visit, so Lille scores could only be calculated for 18 of 19 patients.

Lille scores are used in clinical practice to help determine the prognosis for AH patients after 7 days of treatment. The lower the Lille score, the better the prognosis is for the AH patient. Patients with a Lille score below 0.45 have an 85% 6-month survival rate vs. those with Lille scores of above 0.45, who have only a 25% 6-month survival rate (Louvet A et al., Hepatology, 45:1348-54 (2007)). In the present study, the median Lille score for the 18 AH patients treated with 25HC3S who returned for their Day 7 visit was 0.10. Eighty nine percent (16/18) had a Lille score below 0.45. The median Lille score among the UL cohort of 16 patients treated with either supportive care or supportive care with corticosteroids was 0.41, with 50% (8/16) having a Lille score below 0.45.

In the present trial, the median Lille score of all 18 AH patients treated with 25HC3S from 5 cohorts (both moderate and severe AH) who returned for their Day 7 visit was 0.10, with a quartile range of 0.04 to 0.20. As a comparison, the median Lille score among a cohort of 16 patients treated with supportive care with or without corticosteroid (Standard of Care [SOC]) at the University of Louisville (UL) was 0.41 (shown as the historical control) with a quartile range of 0.14 to 0.73, and 1-month or 3-month mortality rates of 12.5% and 31.3%, respectively. In addition to no treatment emergent serious adverse events observed, patients receiving 25HC3S had significantly lower Lille scores, p=0.005, than those treated with SOC.

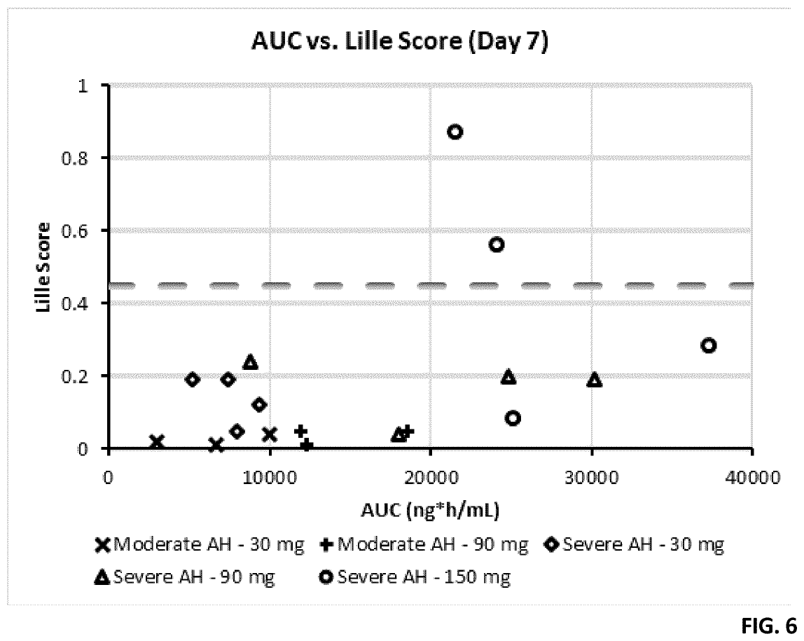

FIG. 6 depicts the Lille scores determined at Day 7 plotted against the AUC of subjects diagnosed with moderate AH that were administered 30 mg and 90 mg of 25HC3S sodium and the Lille scores of subjects diagnosed with severe AH that were administered 30 mg, 90 mg and 150 mg of 25HC3S sodium. As shown in FIG. 6, all subjects administered 30 mg and 90 mg of 25HC3S sodium had Lille scores at Day 7 of less than 0.45. Two of the subjects having severe AH that were administered 150 mg of 25HC3S sodium had Lille scores at Day 7 of greater than 0.45.

The 25HC3S treated AH patients also showed a statistically significant reduction from baseline of serum total bilirubin levels at Day 7, −14% (n=18) as compared to −3% at Day 7 in the UL patients.

The 25HC3S was well tolerated in all patients, with no drug-related serious adverse events reported at any dose level. Drug exposures were dose proportional and were uninfluenced by the severity of the disease.

### Conclusions

In this Phase 2a trial, 25HC3S was well tolerated at the 3 doses tested by all AH patients, including SAH patients. There were no discontinuations, early withdrawal or termination of study drug or study participation due to adverse effects and no serious adverse effects related to 25HC3S were noted. All patients survived through the 28-day follow-up period (i.e., 100% survival rate). The pharmacokinetics of 25HC3S in both moderate AH and severe AH patients was similar for the dose administered and are dose proportional. For a given dose, systemic clearance of 25HC3S is about 5-fold lower in AH patients as compared to healthy subjects.

One or two doses of the drug significantly reduced serum bilirubin levels at Day 7 after treatment and MELD scores at Day 28. There was a 100% treatment response rate (Lille score<0.45) in patients administered 30 mg or 90 mg of 25HC3S and an 89% response rate in all patients. Lille scores of 25HC3S treated patients were significantly better than comparative published historic data.

### Example 2

A randomized, double-blind, placebo-controlled, phase 2b clinical trial evaluating safety and efficacy of 25HC3S in patients with alcoholic hepatitis (AH) will be performed.

### Trial Design

Subjects will be assigned randomly at a 1:1:1 ratio to the following study treatment groups:

| | Optional Corticosteroid |
|---|---|
| Study Treatment (IV infusion) | or Placebo Capsules |
| 25HC3S (30 mg) | Placebo |
| 25HC3S (90 mg) | Placebo |
| Sterile Water for | Methylprednisolone |
| Injection (Placebo) | (32 mg) |

The 1st dose (Day 1 dose) of the assigned IV study treatment (test drug) will be administered in a hospital setting immediately after the subject is randomized.

The second dose of the assigned IV study treatment (test drug) will be given on Day 4, or 3 days after the 1st dose, if the subject is still hospitalized. If a subject meets the discharge criteria prior to Day 4, the subject will receive only one dose of 25HC3S.

A total of no more than two doses of IV infusion study treatment (test drug) will be given.

If the site Investigator elects to use corticosteroids (CS) as part of the standard of care, then the 32 mg methylprednisolone capsules (or matching steroid placebo capsules) should be used. Dosing of the CS capsules or placebo capsules may be, at the decision of the site Investigator based on the AASLD Guidelines, started at any time and also discontinued at any time, especially if their Day 7 Lille score is >0.45.

### Test Drug, Dosage and Mode of Administration

25HC3S sodium salt at 30 and 90 mg (of the 25HC3S free acid) diluted in 100 mL of 5% dextrose or 0.9% sodium chloride and infused over approximately 2 hours.

The sterile ready to use 25HC3S for Injection will be supplied in two sizes:
30 mg/l mL in a single-dose 2 mL glass vial, with 13 mm stopper and crimp seal.
90 mg/3 mL (30 mg/mL) in a single-dose 5 mL glass vial, with 13 mm stopper and crimp seal

The concentration of the DUR-928 Injection product to be used in this study is 30 mg/mL of the 25HC3S free acid, corresponding to 31.4 mg/mL of the 25HC3S sodium salt.

The 25HC3S Injection or placebo will be diluted into a 100 mL infusion bag containing 5% dextrose or 0.9% sodium chloride intravenous solution. The 25HC3S or placebo solution will be administered to the subject by IV infusion over approximately 2 hours.

### Comparator, Dosage and Mode of Administration:

Sterile Water for Injection 3 mL diluted in 100 mL of 5% dextrose or 0.9% sodium chloride and infused over approximately 2 hours.

### Population

Patients with alcoholic hepatitis defined in this clinical trial as:
Onset of jaundice within prior 8 weeks
Average daily consumption of >40 (females) or >60 (males) grams of alcohol for 6 months or longer, with <8 weeks of abstinence before the onset of jaundice. Judgment regarding daily and long-term alcohol use and onset of jaundice will be made by the site investigator.
Serum chemistry (as determined by local laboratory):

$$Serum total bilirubin > 3. mg / dL$$

$$50 < AST < 400 IU / L$$

$$ALT < 400 IU / L$$

$$AST / ALT > 1.5$$

Maddrey's discriminant function≥32 assuming a control prothrombin time of 12 seconds
Model for End-stage Liver Disease (MELD) score: 21-30

### Outcome Measures

### Primary Outcome Measure:

90-day mortality between active group/s and placebo (SOC) group (Time Frame: Day 90)

### Secondary Outcome Measures:

28-day mortality between the treatment groups (Time Frame: Day 28)
Lille score at Day 7 after the initiation of study drug treatment between the treatment groups (Time Frame: Day 7)
MELD score at Day 28 after the initiation of study drug treatment between the treatment groups (Time Frame: Day 28)
ICU days at Day 28 (Time Frame: Day 1 to Day 28)

1. A method of treating alcoholic hepatitis in a human subject in need thereof, the method comprising administering to the subject 5-cholesten-3,25-diol,3-sulfate (25HC3S) or salt thereof in a total amount of about 10 mg to about 400 mg in a period of one month, wherein:
the total amount is sufficient to treat the alcoholic hepatitis;
the total amount is administered in one or more separate doses comprising at least a first dose; and
the period of one month is measured from the beginning of administration of the first dose.

2-4. (canceled)

5. The method of claim 1, wherein the total amount is administered in one dose or in two separate doses, preferably as a single dose.

6. The method of claim 1, wherein at least one of the one or more separate doses comprises from about 10 mg to about 200 mg of 25HC3S or salt thereof.

7. The method of claim 1, wherein at least one of the one or more separate doses comprises from about 20 mg to about 100 mg of 25HC3S or salt thereof.

8. The method of claim 1, wherein the total amount is administered in two or more separate doses that are administered at a dose frequency ranging from daily to once every week.

9. The method of claim 1, wherein:
the total amount is administered in one dose or in two separate doses;
the one dose comprises from about 10 mg to about 200 mg of 25HC3S or salt thereof;
each of the two separate doses comprises from about 10 mg to about 200 mg of 25HC3S or salt thereof; and
the two separate doses are administered at a dose frequency ranging from once every two days to once every four days.

10. The method of claim 1, wherein the administering is performed parenterally, intravenously, intramuscularly, or subcutaneously.

11. The method of claim 1, wherein the administering is performed by injection.

12. The method of claim 1, wherein the 25HC3S or salt thereof is administered in a formulation comprising the 25HC3S or salt thereof and a pharmaceutically acceptable carrier.

13. The method of claim 1, wherein the 25HC3S or salt thereof comprises sodium salt of 25HC3S.

14-15. (canceled)

16. The method of claim 1, wherein the total amount of 25HC3S is administered in from one to five separate doses.

17. The method of claim 1, wherein the total amount of 25HC3S is administered in a single dose.

18. The method of claim 1, wherein each of the separate doses comprises from 10 mg to 100 mg of 25HC3S or salt thereof.

19. The method of claim 1, wherein each of the separate doses comprises from 20 mg to 100 mg of 25HC3S or salt thereof.

20. The method of claim 1, wherein the total amount of 25HC3S or salt thereof is not more than 200 mg.

21. The method of claim 1, wherein:
the total amount is administered in one dose or in two separate doses;
the one dose comprises from 20 mg to 40 mg of 25HC3S or salt thereof;
each of the two separate doses comprises from 20 mg to 40 mg of 25HC3S or salt thereof; and
the two separate doses are administered at a dose frequency ranging from once every two days to once every four days.

22. The method of claim 1, wherein:
the total amount is administered in one dose or in two separate doses;
the one dose comprises from 80 mg to 100 mg of 25HC3S or salt thereof;
each of the two separate doses comprises from 80 mg to 100 mg of 25HC3S or salt thereof; and
the two separate doses are administered at a dose frequency ranging from once every two days to once every four days.

23. The method of claim 1, wherein the human subject has a Model for End-stage Liver Disease (MELD) score of at least 11.

24. The method of claim 1, wherein the human subject has a Maddrey's discriminant function (MDF) of at least 32.
